(19) United States
(12) Patent Application Publication
(10) Pub. No.: US 2009/0293157 A1
(43) Pub. Date: Nov. 26, 2009

(54) PRODUCTION OF PROGENITOR CEREAL CELLS

(75) Inventors: Luke Mehlo, Pretoria (ZA); Zuo-Yu Zhao, Johnston, IA (US)

Correspondence Address: MORRISON & FOERSTER LLP, 12531 HIGH BLUFF DRIVE, SUITE 100, SAN DIEGO, CA, 92130-2040, US

(73) Assignees: CSIR, Pretoria (ZA); Pioneer Hi-Bred International, Inc., Johnston, IA (US)

(21) Appl. No.: 12/358,158

(22) Filed: Jan. 22, 2009

Related U.S. Application Data
(60) Provisional application No. 61/023,012, filed on Jan. 23, 2008.

(51) Int. Cl. A01H 5/00; C12N 5/04; C12N 5/10; C12Q 1/68

(52) U.S. Cl. 800/298; 435/420; 435/419; 435/6

(57) ABSTRACT

A process for the production and maintenance of pluripotent and/or totipotent progenitor cereal cells from undifferentiated callus cells is described. Production of the progenitor cells takes place via direct organogenesis on a medium containing at least one auxin and at least one cytokinin. For example, the auxin may be 2,4-dichlorophenoxyacetic acid, indole-3-acetic acid, picloran, naphthelenacetic acid, indole-3-propionic acid, indole-3-butyric acid, phenyl acetic acid, benzofuran-3-acetic acid or phenyl butyric acid, and the cytokinin may be benzyl amino purine, benzyladenine, thidiazuron, zeatin, isopentyladenine, trans-zeatin or dimethylallyladenine. Processes for transformation of the undifferentiated callus cells and/or the progenitor cereal cells are also described. Typical cereal cells are sorghum, maize, wheat, barley, millet, rye, canola, alfalfa, triticale and rice.

### CROSS-REFERENCE TO RELATED APPLICATIONS

This application claims priority from U.S. provisional application 61/023,012 filed Jan. 23, 2008. The contents of this document are incorporated herein by reference.

### BACKGROUND OF THE INVENTION

This invention relates to a method for the production and maintenance of pluripotent and/or totipotent progenitor cereal cells.

Control of the cell cycle in plants and in animals underpins all in vitro cell and tissue culture systems and is therefore the mainstay of transgenic programs. Founder cells contained in the apical shoot and root meristems of plants are considered equivalents of pluripotent stem cells in animals because they fulfil major criteria used in the molecular definition of stem cells. These criteria include: the property of being clonogenic precursors of daughter cells which remain in the apical shoot tip to replenish the stem cell population (usually about 6-9 cells), or alternatively differentiating during postembryonic stages to grow distal from the shoot tip and form tissues and organs of the entire plant.

In transgenic programs, plant stem cells are of great interest not only because they are pluripotent (i.e. the entire spectrum of all cell types found in the plant can be traced back to stem cells), but because they are also totipotent. As used herein, the term “totipotent” means the unlimited capacity of a single cell to divide and produce all the differentiated cells in an organism. Totipotent cells thus have the capability to regenerate into whole plants.

The concept of stem cells in plants is particularly relevant to Agrobacterium-mediated transformation of sorghum owing to difficulties encountered in establishing efficiently reliable transformation procedures in this crop. Transformation efficiencies are often low, and in the majority of cases, there is a lack of solid evidence to support claims of stable integration of T-DNA. The only reliable and widely used protocol has only recently been established (Zhao et al., 2000). This is perhaps why sorghum is considered relatively recalcitrant, both in terms of tissue culture response and transformability (Zhu et al., 1998).

There are various complex factors influencing T-DNA delivery and regeneration of transgenic sorghum in tissue culture. These include: the sensitivity of sorghum immature embryos to pathogenic influences of Agrobacterium, plant-Agrobacterium cell interactions, factors and molecular activities required for interkingdom macromolecular DNA transfer and sorghum cell cycle-related activities necessary for cell proliferation and subsequent regeneration (McCullen and Binns, 2006). T-DNA transfer to sorghum, and indeed to other previously “difficult to transform” cereals like barley, corn and wheat is no longer limiting, but hypersensitive necrotic response of tissues, particularly in sorghum, is a drawback to the maintenance of transgenic callus and the regeneration of plants (Carvalho et al., 2004; Hansen, 2000). This is probably because many pathogenic bacteria, as is the case with Agrobacterium tumefaciens, possess hypersensitive reaction and pathogenicity (hrp) genes. When these genes are triggered, they elicit a plant defensive, but unfortunately fatal, hypersensitive reaction in the affected cells in an attempt to limit and contain the infection.

There is therefore an ongoing need for a method to produce pluripotent and/or totipotent progenitor cereal cells, particularly sorghum, at high frequency.

Currently the prior art is silent regarding the use of undifferentiated cereal callus cells for the hormonally-induced, enriched production of pluripotent and/or totipotent progenitor cells for long-term maintenance in the callus phase and as a substrate for Agrobacterium-mediated transformation for the generation of cloned cereal cells and the subsequent generation of transgenic cereal plants.

### BRIEF SUMMARY OF THE INVENTION

According to a first aspect to the present invention there is provided a process for the production of pluripotent and/or totipotent progenitor cereal cells, the process comprising the steps of:
selecting a population of cells including undifferentiated cereal callus cells; and
culturing the undifferentiated cereal callus cells in a primary plant tissue culture medium containing at least one auxin and at least one cytokinin to produce pluripotent and/or totipotent progenitor cereal cells.

At least a portion of the undifferentiated cereal callus cells may be converted to pluripotent and/or totipotent progenitor cereal cells in the culture medium, and the progenitor cells may be multiplied at a greater rate than non-progenitor cells.

The undifferentiated cereal callus cells may be selected from a cereal plant such as sorghum, maize, wheat, barley, millet, rye, canola, alfalfa, triticale and rice, and more particularly from scutellum tissue of the plant. The scutellum tissue may be from an embryo, and in particular from a zygotic embryo (mature or immature)

The undifferentiated cereal callus cells may be cultured in the primary tissue culture medium for a period of from about 10 days to about 4 weeks, more particularly from about 14 to about 21 days, and even more particularly about 15 days. The pluripotent and/or totipotent progenitor cereal cells formed during the culture period may organize into cell aggregates to form shoot apical meristematic domes and primordial shoots by a process of direct organogenesis.

The undifferentiated cereal callus cells may be obtained from plant tissue that has already undergone a transformation step to transform the plant tissue with an homologous or heterologous gene. Alternatively, the process may include an additional step of transforming the pluripotent and/or totipotent progenitor cereal cells with an homologous or heterologous gene. The transformation step may be Agrobacterium-mediated, such as with A. tumefaciens, or may be via biolistic bombardment.

The pluripotent and/or totipotent progenitor cereal cells formed in the primary tissue culture medium may be maintained in a state of perpetual proliferation, with the primary tissue culture medium being replaced as needed. In this way, pools of transgenic cereal cells may be maintained indefinitely.

When it is desired to regenerate plantlets, the pluripotent and/or totipotent progenitor cereal cells may be moved to a secondary plant tissue culture medium. The secondary plant tissue culture medium may include at least one cytokinin and optionally at least one auxin.

The cytokinin in the primary or secondary tissue culture medium may be benzyl amino purine, benzyladenine, thidiazuron, zeatin, isopentyladenine, trans-zeatin or dimethylallyladenine or combinations thereof.

The auxin in the primary or secondary tissue culture medium may be 2,4-dichlorophenoxyacetic acid, indole-3-acetic acid, picloran, naphthelenacetic acid, indole-3-propionic acid, indole-3-butyric acid, phenyl acetic acid, benzofuran-3-acetic acid or phenyl butyric acid or combinations thereof.

The auxin and the cytokinin may be present in the culture medium in a ratio of about 1:4.

The transformation frequency obtained by the process may be at least 5%, at least 10%, at least 15%, at least 20%, or at least 30%. More particularly, the transformation frequency may be at least 19%.

According to a second embodiment of the invention, there is provided a process for producing transgenic cereal cells, the process comprising the steps of:
transforming cereal tissue;
selecting from the transformed cereal tissue a population of cells including undifferentiated cereal callus cells; and
culturing the undifferentiated cereal callus cells in a primary plant tissue culture medium containing at least one auxin and at least one cytokinin to produce pluripotent and/or totipotent progenitor cereal cells.

Further embodiments of the invention include pluripotent and/or totipotent progenitor cells, transformed cells and transgenic plant parts, plantlets or plants produced by the processes substantially as described above.

### BRIEF DESCRIPTION OF THE DRAWINGS

The invention will now be described with reference to the following figures:

FIG. 1: Schematic diagram of the plasmid PHP15303 used for Agrobacterium transformation. This plasmid contains the visual marker, gfp gene driven by the maize Ubiquitin promoter and the selectable marker, bar gene driven by the 35S promoter. UBI1ZMPRO=Maize Ubiquitin promoter; UBI1ZMINTRON=maize ubiquitin 1 intron; GFPM-EXON1 & 2=exon 1 or 2 for green fluorescence gene; PINII TERM=pin II terminator sequence; CAMV35S ENH=Cauliflower mosaic virus 35S enhancer sequence; CAMV35S PROM=Cauliflower mosaic virus 35S promoter; ADH1 INTRON1=Alcohol dehydrogenase intron 1 sequence; BAR=selectable marker bar gene for phosphinothricin (PPT) resistance. RB=right boarder sequence for Agrobacterium tumefaciens; LB=left boarder sequence for Agrobacterium tumefaciens.

FIG. 2: Enriching undifferentiated sorghum callus for competency towards totipotency, pluripotency and efficient regeneration. (A): Sorghum callus cultured for 15 days on 710B; (B-H): Differential stages of sorghum callus development towards miniscule shoot organogenesis after culture for 15 days on modified 710B (2 mg/l BAP+0.5 mg/l 2,4-D. (H) is a close-up of the callus unit in (F) showing defined clusters/aggregates of apical shoot meristematic domes and leaf primordia in later stages of development. Each one of the embryoids shown in (G) or domes in (H) has an inherent potential to regenerate other meristematic domes at an exponential rate and each one of these meristematic tissues has the potential to regenerate a plant.

FIG. 3: Enriching organised maize meristematic tissue cells for stem cells. A: An isolated organized maize apical shoot segment showing the development of apical shoot meristematic domes (containing shoot meristem stem cells) after 3 weeks culture on MSC2 (see Table 1.0). B: Establishment of a virtual lawn of multiple shootlets from the tissue in (A) after 4-5 weeks. C: when the shootlets in (B) are exposed to light and on medium MSCSP (Table 1.0), green shoots regenerate. D: The two rows on the left represent maize plants developed from (C) whereas the two rows on the right represent maize plants germinated and grown from seed of the same genotype.

FIG. 4: Enrichment for organogenesis in sorghum immature embryo-derived callus. Actual size=1 cm in diameter. This 30-day old embryogenic callus mass was derived from a single immature sorghum embryo cultured on 710B+2 mg/l BAP+0.5 mg/l 2,4-D in the dark at 28° C. A total of 99 plants were derived from multiple shoots developed from this tissue.

FIG. 5: Schematic representation of current standard transformation procedures of sorghum transformation; FIG. 5A illustrates sorghum panicles, their transformation to immature embryos, and an immature embryo-derived Type I callus that can be subjected to selection; and FIG. 5B illustrates sorghum panicles, their transformation to immature embryos, and an immature embryo-derived callus highly enriched for organogenesis that can then be selected to derive pools of trangenic tissues.

FIG. 6: Sorghum immature embryo-derived callus can acquire cellular competence for maintenance of transgenic cell pools and high frequency regeneration. Horizontal rows of images linked by a black line on the left of the figure are light microscope (top row) and GFP expressing (bottom row) mirror images of pools of transgenic cell aggregates, multiple shoot meristematic primordia and apical meristematic shoot tissues. These pools of transgenic tissues have been subjected to PPT selection for over 60 days (thus reflecting stable integration) and were derived from the inclusion in the culture system of a stem cell/pluripotent/or totipotent enrichment phase using 2 mg/l BAP and 0.5 mg/l 2,4-D as explained in the materials and methods. Dark areas as shown by a white arrow indicate necrotic non-transformed cells and tissues killed by PPT selection. White sectors (bottom rows) also indicated by a white arrow show sectors of GFP expressing stably integrated gfp gene. These images provide proof that this technique can be utilized to accumulate and maintain a perpetual pool of transgenic cell lines.

FIG. 7: High efficiency regeneration of multiple shoots of sorghum derived from direct organogenesis of sorghum immature embryo-derived callus after enrichment for stem cell, pluripotency and totipotency. A=Masses of multiple shoots and, B=Multiple shoots split into smaller units. These shoots were obtained after 3 weeks in the dark and are ready for exposure to light. Over 99 transgenic plantlets could be regenerated from a single transformed immature embryo.

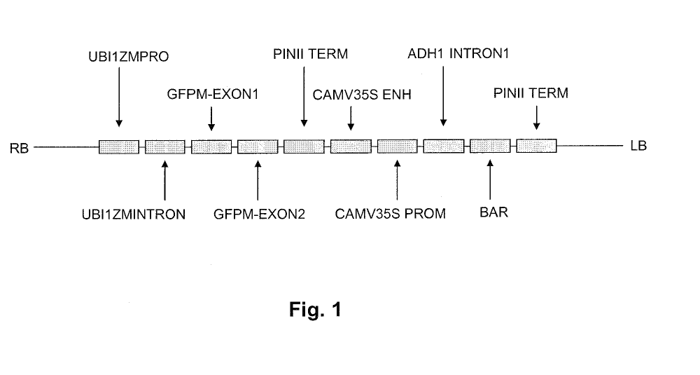

### DETAILED DESCRIPTION OF THE INVENTION

A process that enriches undifferentiated sorghum callus cells for highly pluripotent and totipotent progenitor cells through the use of auxins and cytokinins is described herein.

Agrobacterium-mediated transformation of sorghum consistently yields low transformation frequencies, on average less than 3%.

The applicants have shown that T-DNA transfer into sorghum cells is not essentially the problem when using the broad-spectrum super binary vectors, for example those from Japan Tobacco. Instead, it has been identified that cell survival and regeneration after Agrobacterium infection and T-DNA transfer is the biggest challenge in sorghum transformation. Cell survival is compromised by phenolic compounds that are produced by sorghum embryos due to wounding and also due to the burden of infection by Agrobacterium. Similarly, cell death and necrosis result from Agrobacterium's pathogenic elicitation of the hypersensitive response genes in infected cells which effectively kills the cells in a bid to activate a defensive mechanism (Hansen, 2000). The slower cell cycle in sorghum also leads to poor survival and regeneration, especially in cultures that have been kept on selection over extended periods of time. Given, therefore, that transformation yields few transformed cells compared to untransformed cells, a further challenge is that of maintaining the few transgenic cell lines in a proliferative state that would lead to regeneration.

Integrating this process into transformation protocols results in regeneration and transformation frequency being raised from an average of less than 3% to an average of 19% of transformed immature embryos.

The present process utilizes direct organogenesis from undifferentiated callus cells to multitudes of organized functional apical shoot meristems in sorghum transformation protocols, to yield previously unreported transformation frequencies as high as 19%. This is the first time such a process has been described. This high transformation frequency can be ascribed to an increased maintenance of transgenic cell pools in a robust state of cell division and to the conversion of undifferentiated callus cells into multitudes of pluripotent and highly totipotent progenitor cells. Direct organogenesis has previously been reported in maize, finger millet, Gaetn and crowfoot grass for the development of multiple shoots. However, organogenesis was achieved in these crops through culturing isolated shoot apices (already organized tissues) and not through undifferentiated callus cells as is the case in the process of the present application.

Other investigators have addressed the issue of poor cell survival post-transformation through other techniques. Visual selection has previously been used, and the green fluorescent protein (GFP) has been employed to select for transgenic cells instead of antibiotics or herbicide selective agents which kill untransformed cells.

Shorter subculture intervals have also been advocated after the realization that phenolic compounds produced by embryos following Agrobacterium infection are detrimental to cell survival (Zhao et al., 2000).

Similarly, use of alternative and less harsh selective strategies such as the phosphomannose isomerase system in which untransformed cells are not necessarily killed, but inhibited from growing at the expense of transformed cells, have also been explored in sorghum transformation.

Despite all these strategies, transformation frequencies when Agrobacterium tumefaciens is used are still low, particularly in sorghum (less than 3%), owing to other suboptimal, but critical factors intrinsic to Agrobacterium-mediated delivery systems, for example: genotype dependency, Agrobacterium strains, plasmid vectors, virulence gene-inducing compounds, medium compositions and a host of other plant tissue-specific factors.

This process involves the recruitment and conversion of somatic cells in Type I scutelum-derived callus of sorghum in less than about 15 days to pluripotent and highly totipotent progenitor cells (equivalent of stem cells in animals), which otherwise are only resident in organized apical meristems (shoots and roots) in plants, and only number about 6-9 cells/meristem in the said natural niches. During this short culture period of about 15 days, the newly converted progenitor cells organize into cell aggregates to form shoot apical meristematic domes and primordial shoots which are highly totipotent and can be efficiently regenerated into complete plantlets within an additional one to four weeks subsequent to the initial culture period of 15 days. Although a culture period of 15 days is exemplified herein (during which period are formed), it will be apparent to a person skilled in the art that hundreds or even thousands of copies could be formed in a culture period of about 3 to 4 weeks.

The process can be used to enrich for pools of transgenic cells from rare transformation events. This can be achieved through perpetual proliferation and increased regeneration frequency even in “tired” cell cultures that have undergone diminished totipotency owing to extended culture periods. Long culture periods are common in transformation systems and are designed to ensure effective selection of transformed cells from untransformed cells. This is usually achieved through the use of herbicides (e.g. PPT), antibiotics (e.g. hygromycin, kanamycin) or other selection agents/mechanisms, for example phosphomannose isomerase selection system.

By enriching for rapid cell division through the use of the disclosed method, and in particular when applied to morphogenetically flexible progenitor cells, it has been possible to partially overcome sorghum cell death and the deleterious effects of phenolic compounds that are a common phenomenon in infected immature embryos of sorghum, and cell necrosis often caused by Agrobacterium's pathogenic elicitation of the hypersensitive response in plant cells.

The process of the present invention significantly improves transformation frequency from the current low levels of less than 3% to about 19% and even higher through enrichment for cells that are vigorously competent in cell division, pluripotency and totipotency. Plant regeneration cycles are also shortened. A further aspect of this invention is that it is equally applicable to other cereal crops, such as corn, rice, barley, wheat and millets. This is evident from the fact that, in implementing the method of the present invention on sorghum, a highly transformable corn genotype, GS3, was often used as a control in optimizing transformation parameters. This observation and extended application to other elite crops is in line with the fact that the step of enriching for pluripotent and totipotent progenitor cells is compatible with and can be conveniently inserted into current protocols of transformation, whether it be sorghum, maize (corn), rice, wheat, barley, millet, rye, canola, alfalfa, triticale and the like, and is independent of method of transformation. This technique is also ideal in implementing high throughput transformation systems.

Suitable cytokinins for use in one or more of the tissue culture media used in the process include benzyl amino purine, benzyladenine, thidiazuron, zeatin, isopentyladenine, trans-zeatin or dimethylallyladenine or combinations thereof, while suitable auxins for use in one or more of these tissue culture media include 2,4-dichlorophenoxyacetic acid, indole-3-acetic acid, picloran, naphthelenacetic acid, indole-3-propionic acid, indole-3-butyric acid, phenyl acetic acid, benzofuran-3-acetic acid or phenyl butyric acid or combinations thereof.

The present invention is further described by the following examples. Such examples, however, are not to be construed as limiting in any way either the spirit or scope of the invention.

### EXAMPLES

### Plant Materials and Media Compositions

The sorghum public line, P898012 (originally supplied to Pioneer Hi-Bred International-USA by Dr. John Axtell, Purdue University; see Zhao et al., 2000) and the maize genotype denoted GS3 (developed by Pioneer Hi-Bred International-USA) were used for the isolation of immature zygotic embryos at 9-14 days after pollination. The two genotypes were grown in Pioneer Greenhouses primarily as described (Zhao et al., 2000). Sterilization of sorghum panicles and corn ears was carried out with 50% Chlorox Bleech (3.075% (v/v) sodium hypochlorite) and 0.1% (v/v) Tween 20 for 20 minutes and then rinsed three times with sterile distilled water. This sterilization procedure was repeated with 10% Chlorox bleech (0.615% (v/v) sodium hypochlorite). Immature zygotic embryos ranging in size from 0.8 mm-1.8 mm were isolated and treated as indicated in the transformation procedures outlined below. The compositions of various media used in this study are outlined in Table 1.

TABLE 1
Media Composition

| Media and usage | Composition |
|---|---|
| 700: | The following components were dissolved |
| Liquid media used | sequentially in 950 ml polished de-ionized water: |
| for Agrobacterium | 4.3 g MS basal salt mixture; 0.1 g Myo-Inositol |
| infection of | (10000X); 0.5 ml Nicotinic acid (1 mg/ml stock); |
| immature embryos | 0.5 ml Pyridoxine (1 mg/ml stock); 2.5 ml Thiamine |
| (GS3, P898012) | HCl. (4 mg/ml); 1 g Vitamin Casamino acids; 68.5 g |
| | Sucrose; 36 g glucose |
| | PH adjusted to 5.2 with 1M KOH. |
| | Final volume adjusted to 1 L with polished de- |
| | ionized water |
| | The media filter sterilized through a 0.22 m filter |
| | and aliquoted into 12 ml volumes and stored at 4° C. |
| | Quality control tests carried out by streaking a few |
| | microlitres of the media onto microbial plates to |
| | check for contamination over 3 days. |
| 710B: | The following components were dissolved |
| Co-cultivation | sequentially in 950 ml polished de-ionized water: |
| medium | 4.3 g MS basal salt mixture; 0.1 g Myo-Inositol; 0.5 |
| | ml Nicotinic acid (1 mg/ml stock); 0.5 ml |
| | Pyridoxine (1 mg/ml stock); 2.5 ml Thiamine HCl. |
| | (4 mg/ml); 4 ml 2,4-D (0.5 mg/l stock); 20 g Sucrose; |
| | 10 g glucose; 0.7 g L-proline; 0.5 g MES buffer. |
| | PH adjusted to 5.8 with 1M KOH. |
| | Final volume adjusted to 1 L with polished de- |
| | ionized water |
| | 4 g Sigma agar added |
| | Autoclaved and cooled to 45-55° C. |
| | Add 1 ml (100 mM stock) filter sterilized |
| | acetosyringone |
| | Add 1 ml (10 mg/ml) Ascobic acid |
| | Mix and pour plates |
| | Quality control tests carried out by streaking a few |
| | microlitres of the media onto microbial plates and |
| | incubating at 28° C. to check for contamination over |
| | 3 days. |
| 720J: | The following components were dissolved |
| First two weeks | sequentially in 950 ml polished de-ionized water: |
| PPT selection (for | 4.3 g MS basal salt mixture; 0.5 ml Nicotinic acid |
| transformations | (1 mg/ml stock); 0.5 ml Pyridoxine (1 mg/ml stock); |
| carried out with the | 2.5 ml Thiamine HCl. (4 mg/ml); 0.1 g Myo- |
| bar gene) | Inositol; 3 ml 2,4-D (0.5 mg/l stock); 20 g Sucrose; |
| | 0.7 g L-proline; 0.5 g MES buffer. |
| | PH adjusted to 5.8 with 1M KOH. |
| | Final volume adjusted to 1 L with polished de- |
| | ionized water |
| | 4 g Sigma agar added |
| | Autoclaved and cooled to 60° C. |
| | 1 ml added of Ascobic acid (10 mg/ml) |
| | 2 ml Agribio carbenicillin (50 mg/ml) added |
| | 5 ml PPT (10 mg/ml Glufosinate —NH₄) |
| | Mix and pour plates |
| | Quality control carried out by streaking a few |
| | microlitres of the media onto microbial plates and |
| | incubating at 28° C. to check for contamination over |
| | 3 days. |
| 720K | Essentially similar to 720J except that 10 ml PPT |
| | (10 mg/ml Glufosinate —NH₄) was used instead of 5 |
| | mg/l PPT |
| 289J | The following components were dissolved |
| | sequentially in 950 ml polished de-ionized water: |
| | 4.3 g MS basal salt mixture; 1.0 g Myo-Inositol; 5 |
| | ml of MS Vitamin stock solution; 1 ml zeatin (of |
| | stock 0.5 mg/ml); 0.7 g L-Proline; 60 g sucrose; |
| | PH adjusted to 5.6 with 1M KOH. |
| | Final volume adjusted to 1 L with polished de- |
| | ionized water |
| | 4 g Sigma agar added |
| | Autoclaved and cooled to 60° C. |
| | After autoclaving add; 2.0 ml of IAA (0.5 mg/ml |
| | stock); 1.0 ml ABD (0.1 mM stock); 0.1 ml of |
| | Thidiazuron (1.0 mg/ml stock); 2.0 ml carbenicillin |
| | (50 mg/ml stock); 5.0 ml PPT (1.0 mg/ml stock of |
| | Glufosinate-NH4). |
| | Mix and pour plates |
| | Quality control carried out by streaking a few |
| | microlitres of the media onto microbial plates and |
| | incubating at 28° C. to check for contamination over |
| | 3 days. |
| 289J#1 | 289J + [0.5 mg/l BAP + 0.5 mg/l IBA + 5 mg/l PPT], |
| | Remove [zeatin, IAA, ABA, TDZ] |
| 289J#2 | 289J + [0.5 mg/l BAP + 0.5 mg/l IBA + 5 mg/l PPT], |
| | Remove [zeatin, IAA, ABA] (Note TDZ is |
| | included, as opposed to 289J#1 above) |
| 289J#3 | 289J + [1 mg/l BAP + 5 mg/l PPT], Remove [zeatin, |
| | IAA, ABA, TDZ] |
| 289J#4 | 289J + [2 mg/l BAP + 0.5 mg/l NAA + 5 mg/l PPT], |
| | Remove [zeatin, IAA, ABA, TDZ] |
| 289J#4 | 289J + [0.5 mg/l 2,4-D + 10 mg/l BAP + 5 mg/l PPT], |
| | Remove [IAA, ABA, TDZ] |
| UCB | Per litre: 4.3 g MS basal salt mixture; 0.25 g Myo- |
| | Inositol; 1.0 g Casein hydrolysate; 0.5 mg BAP; 10 |
| | mg Thiamine-HCl; 1.0 mg 2,4-D; 30 g maltose; |
| | 0.69 g L-proline; 0.0049 M CuSO₄; Ph 5.8 adjusted |
| | with KOH; 3.5 g phytagel |
| MSC1 | Per litre: MS basal medium (+macro and |
| | micronutrients + vitamins), 2 mg BAP, 500 mg |
| | casein hydrolysate (CH), 2.5 g gelrite gellan gum; |
| | plus or minus 100 mg/l carbenicillin |
| MSC2 | Per litre: MS basal medium, 2 mg BAP, 0.5 mg 2,4- |
| | D, 500 mg CH; plus or minus 100 mg/l carbenicillin |
| MSC2P | Per litre: MSC2 (—CH), 3 mg Glufosinate |
| | ammonium; plus or minus 100 mg/l carbenicillin |
| MSCSP | Per litre: MS basal medium, 0.5 mg BAP, 0.5 mg |
| | IBA, 3 mg Gluphosinate ammonium; plus or |
| | minus 100 mg/l carbenicillin; plus or minus 100 mg/l |
| | carbenicillin |
| MSCRP | Per litre: MS basal medium, 1 mg IBA, 3 mg |
| | Gluphosinate ammonium; plus or minus 100 mg/l |
| | carbenicillin |

### Transformation Procedures and Identification of Putative Positive Transformants

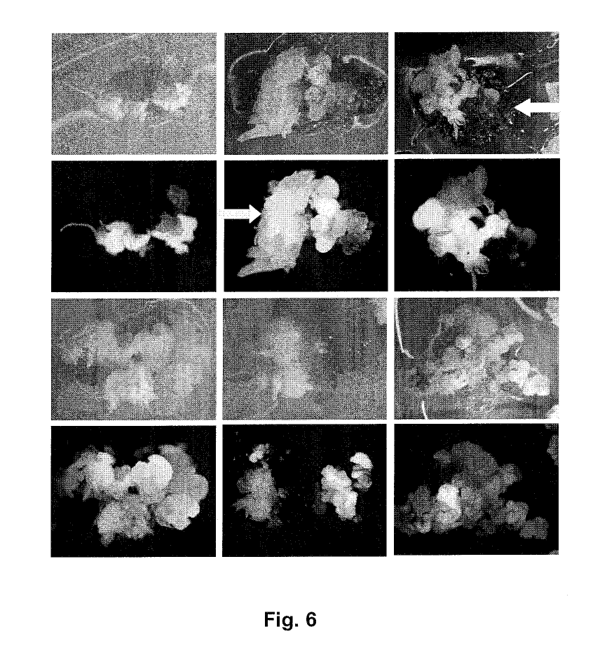

Agrobacterium tumefaciens
Transformation was carried out in 6 distinctive but sequential phases. The medium used at each phase is given in Table 1.
1. Freshly isolated embryos of P898012, GS3 or TRX were mixed into 1.5 mL of medium 700 either lacking or containing 100 mM acetosyringone. The concentration of A. tumefaciens harbouring the vector PHP15303 (FIG. 6) in the suspension was adjusted to a range between 0.857×10⁹ cfu/mL [Optical Density (OD) approximately. 0.6 at 550 nm] and 0.5×109 cfu/mL (OD=0.35 at 550 nm). The infection suspension was vortexed gently for 15 seconds, poured into 1 cm-diameter microplates and vacuumed for 5 minutes with gentle rocking for mixing.
2. The Agrobacterium suspension was then aspirated and the embryos plated on co-cultivation medium 710B either lacking or containing 100 mM acetosyringone for 3 days (co-cultivation) and cultured in the dark at 25° C.
3. After the 3-day co-cultivation, the embryos were transferred onto resting medium 710B containing 100 mg/mL carbenicillin, an antibiotic to kill off the Agrobacterium. This medium did not contain acetosyringone. The embryos were cultured in the dark for 4 days at 28° C. during this phase.
4. The embryos were either transferred onto medium 720J or 720J containing 2 mg/L BAP alone or 720J containing 2 mg/L BAP and 0.5 mg/L 2,4-D for two weeks.
5. The proliferating embryos were then subjected to a second phase of selection on either medium 720K or 720K containing 2 mg/L BAP alone or 720K containing 2 mg/L BAP and 0.5 mg/L 2,4-D until putative transgenic callus units averaging about 1 cm in diameter were observed.
6. Putative transgenic calli were regenerated on either medium 289J or modifications outlined in the transformation scheme below:

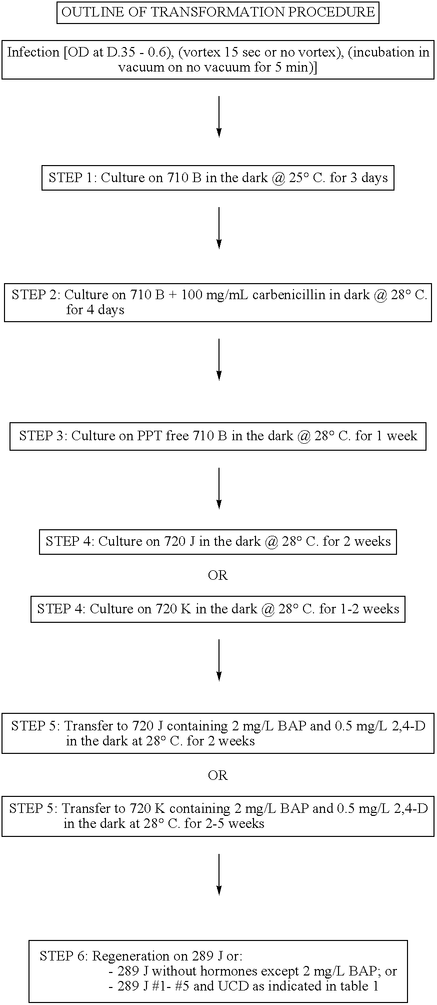

Fresh subcultures were conducted at 1-2 week intervals depending on the amount of observable phenolic compounds on the medium. Putative transgenic calli from one embryo were kept separate and tentatively treated as one event until proven through analysis to contain more than one event. This can be performed by analysing Southern hybridization integration patterns of each regenerated plant.

Transformation of GS3 or TRX maize immature embryos was carried out in a similar manner to sorghum and cultured on medium identical to that for sorghum but additional media were also used in the following manner:
MSC1 in place of 710B;
MSC2 in place of 710B;
MSC2P in place of the selection media 720J or 720J and the respective modifications;
MSCSP in place of 289J and its shoot regeneration modifications; and
MSCRP for root regeneration from developed meristematic tissues and shoots.

### Biolistics-Mediated Transformation of Sorghum Immature Embryos

Immature embryos of sorghum isolated as described in previous sections were cultured on callus initiation medium (see Tables 2 and 3) for 3-8 days at 28° C. in the dark before bombardment. After this initial culture period, the embryos were cultured on osmoticum media (callus initiation medium described in Table 2 and 3 containing 0.2 M sorbitol+0.2 M mannitol sugars) for 3-4 hours.

TABLE 2
Additional media compositions used for particle bombardment

| | L3 Macro-nutrients stock | |
|---|---|---|
| L3 Macro-nutrients | quantities (g/L) | Usage |
| KNO₃ | 35 | Use 50 ml of L3 stock solution |
| NH₄NO₃ | 4 | per litre of medium |
| MgSO₄•7H₂O | 7 | |
| KH₂PO₄ | 4 | |
| CaCl₂•2H₂O | 9 | |
| | L3 Micro-nutrients stock | |
| L3 Micro-nutrients | quantities (g/L) | Usage |
| H₃BO₃ | 1.0 | Use 5 ml of L3 Micro- |
| MnSO₄•4H₂O | 5.0 | nutrients stock solution per |
| ZnSO₄•7H₂O | 1.5 | litre of medium |
| NaMoO₄•2H₂O | 0.050 | |
| CuSO₄•5H₂O | 0.0050 | |
| CoCl₂•6H₂O | 0.0050 | |
| KI | 0.150 | |
| | Fe-source stock | |
| Fe-Source | quantities (g/L) | Usage |
| FESO₄•7H₂O | 2.78 | Use 10 ml of Fe-source stock |
| Na₂EDTA•2H₂O | 3.73 | solution per litre of medium |
| | G2 Vitamin Stock | |
| G2 Vitamins | quantities (g/L) | Usage |
| Thiamine-HCL | 2.0 | Use 5 ml of Vitamin stock |
| Pyridoxine-HCL | 0.2 | solution per litre of medium |
| Nicotinic acid | 0.2 | |
| myo-inositol | 20 | |
| L-glutamine | 84 | |

TABLE 3
Media used for sorghum tissue culture
in combination with particle bombardment

| | | | | | Second Phase | | Third Phase | |
|---|---|---|---|---|---|---|---|---|
| | Callus | | First phase | | selection: | | selection: | |
| Nutrient | Initiation | | selection | | maturation | | Regeneration | |
| L3 Macro- | + | | + | | + | | + | |
| nutrients | | | | | | | | |
| L3 Micro- | + | | + | | + | | + | |
| nutrients | | | | | | | | |
| Fe-source | + | | + | | + | | + | |
| G2 Vitamins | + after | | + after | | + after | | + after | |
| | autoclaving | | autoclaving | | autoclaving | | autoclaving | |
| L-Proline | 20 mM after | | | | | | | |
| | autoclaving | | | | | | | |
| 2,4-D | 2.5 | mg/l | | | | | | |
| Kinetin | | | | | | | 0.5 | mg/l |
| IAA | | | | | | | 0.2 | mg/l |
| Mannose | | | 9 | g/l | 9 | g/l | 9 | g/l |
| Maltose | | | 12 | g/l | 24 | g/l | 12 | g/l |
| pH | 5.8 | | 5.8 | | 5.8 | | 5.8 | |
| Gelrite | 4 | g/l | 4 | g/l | 4 | g/l | 4 | g/l |

Some components (indicated by “+”) and their exact quantities are derived from Table 2.

Following this culture on osmoticum medium, particle bombardment was then carried out according to the scheme outlined below:
30 mg of 0.6 μm gold particles sterilized by vortexing for 5 minutes in 70% (v/v) ethanol, and then pelleted by spinning at 10 000 rpm for 5 seconds: repeat 3×;
1 mL of sterile distilled water used to wash particles through vortexing in a similar manner to the ethanol wash above: repeat 3×; and
After pelleting and discarding the wash water, 500 μl of sterile glycerol was then added to the particles.

Coating of the gold particles with linear fragments of plasmid DNA (pABS042 and pABS044; and linear fragment of plasmid pNOV3604 containing the PMI selectable marker gene) was carried out essentially as described by McCabe and Christou, (1993) except that the final bombardment volume of 6 μl contained 90 ng of the PMI selectable gene and 70 ng of the target gene (in this experiment these were either pABS042 or pABS044 linear fragments).

### Briefly:

Macrocarrier gold particles were mixed with DNA and the mixture vigorously vortexed for 4 seconds;
Calcium chloride was added to the DNA/gold mixture and a brief vortex carried out for a further 5 seconds;
Spermidine was then added, a single drop at a time whilst the mixture was gently vortexed to ensure uniform coating;
The mixture was pulse spun for 3 seconds and the supernatant discarded;
70% (v/v) ethanol was used to wash the mixture by brief vortex and the wash discarded;
A similar wash was carried out with absolute ethanol;
The final suspension was carried in absolute ethanol; and
The Biolistic PDS-1000/He Biorad system was used for bombardment according to recommendations from the manufacturer.

Following the bombardments, the sorghum embryos were plated on callus initiation medium (Tables 2 and 3) and cultured in the dark for 7 days at 28° C. This was followed by transfer to first phase selection medium (Tables 2 and 3) for 4 weeks in the dark at 28° C. The following scheme was adopted for all subsequent subcultures and transfers:
Transfer to second phase selection (Tables 2 and 3) for three-four weeks in the dark at 28° C., or to similar second phase selection medium without any other hormones besides (2 mg/L BAP+0.5 mg/L 2,4-D) or (2 mg/L BAP alone).
Transfer to third phase selection: regeneration medium (Table 2 and 3) for 2-3 weeks in the dark at 28° C. or to similar third phase selection medium: regeneration without any other hormones besides (2 mg/L BAP+0.5 mg/L 2,4-D) or (2 mg/L BAP alone) until fully grown plantlets could be transferred to the greenhouse for hardening off.

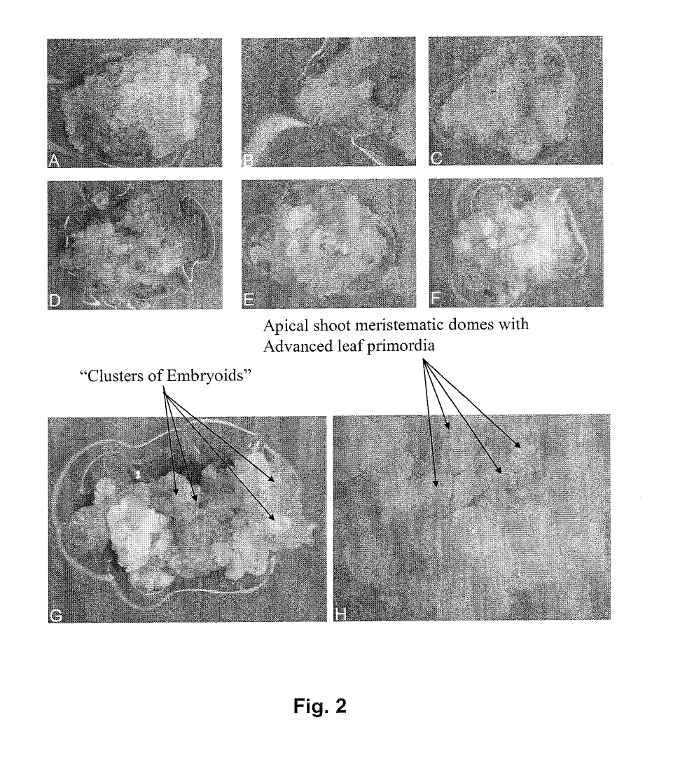

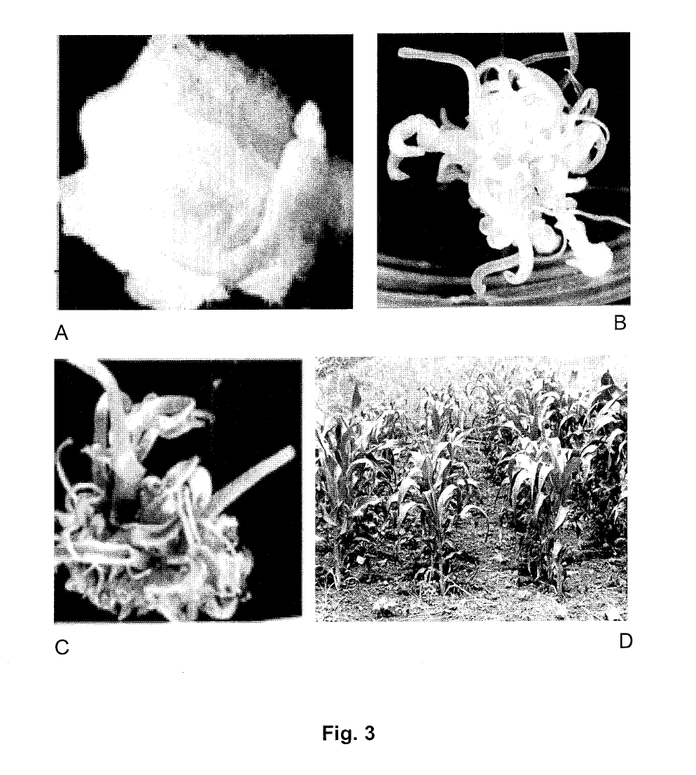

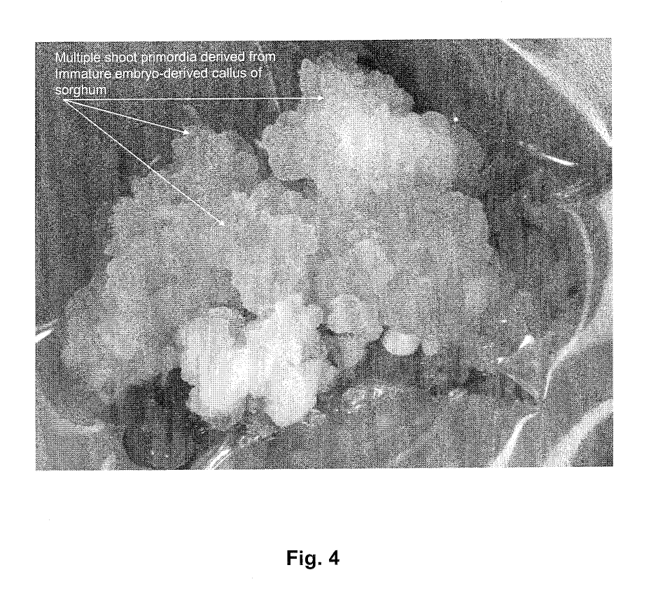

The process of direct organogenesis and enrichment for progenitor stem cells shown in FIGS. 2 and 4 is a unique process involving scutellum-derived callus as the starting material. To distinguish this process from direct organogenesis involving the starting material as an already organized tissue, maize apical shoot tips containing the apical shoot meristem were used to enrich for progenitor stem cells and eventually multiple shoots. In this case, apical shoot tips derived from aseptically germinated mature seeds of maize were cultured on medium containing similar amounts of hormones (2 mg/l BAP and 0.5 mg/l 2, 4-D) to obtain multiple shoots (FIG. 3).

Undifferentiated sorghum and maize callus, derived from scutellum tissue of immature zygotic embryos, can be enriched for competency towards developing pluripotent and totipotent progenitor stem cells at very high frequency within a short period of about 15 days. These progenitor cells can then be redirected towards differentiating miniscule apical shoot meristems and multiple shoots in a novel process of direct organogenesis (callus developing directly to shoots). The processes involved in this enrichment technique occur at near exponential rate, with each apical meristematic dome capable of producing many more apical meristematic domes. Because each apical meristematic dome has the potential to form an individual plant, the number of plantlets that can be derived from this novel enrichment technique is substantial (FIG. 2).

The process of enrichment for pluripotent progenitor stem cells in sorghum callus illustrated in FIGS. 2 and 3 is accompanied by a concomitant enrichment of tissues for totipotency as well. Organized shoot primordia can be developed from such tissues at high frequency, indicating that this enrichment phase can be an invaluable component of transformation systems in difficult-to-transform and regenerate elite crops. Further, the enrichment for pluripotency and totipotency can be ideal for high throughput transformation systems where large numbers of transgenic plants are desired. Masses of organized multiple shoot primordia can be seen in FIG. 4.

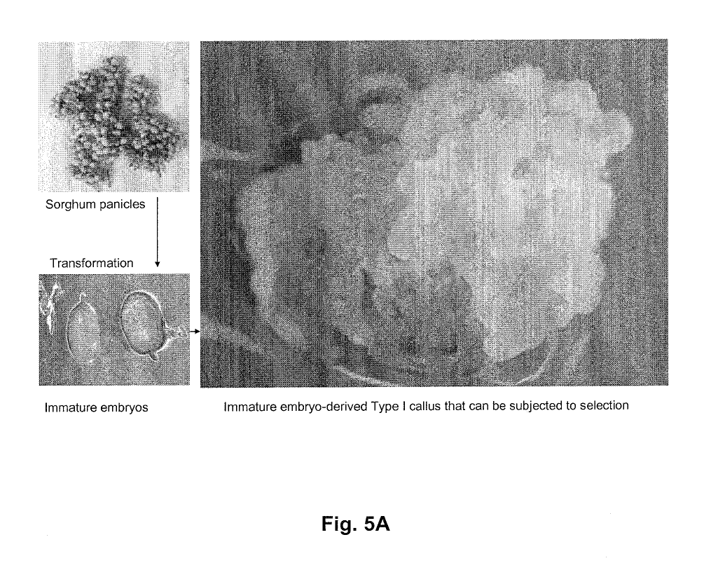

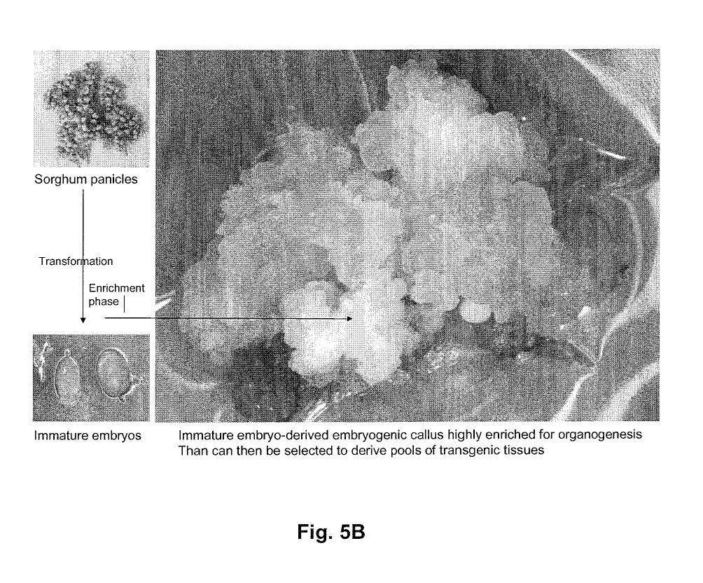

Based on the results obtained in FIGS. 2 to 4, a new scheme of transformation of sorghum was devised. Current protocols relying on methods developed by Zhao et al. (2000) are depicted in FIG. 5a, whereas a protocol based on results of success with enrichment for pluripotent and totipotent progenitor stem cells is shown in FIG. 5b.

Sorghum immature embryo-derived callus can acquire cellular competence for recruitment and maintenance of transgenic cell pools and high frequency regeneration. Green Fluorescence Protein (GFP) was used to image and track down the transformation process of sorghum immature embryo-derived callus cells to stable DNA integration and the development of transgenic multiple shoots. These transgenic multiple shoots were derived from pools of transgenic cell lines that have conferred selective advantage owing to them taking up the bar gene in addition to the gfp gene. The images in FIG. 6 show that the technique of enriching for pluripotent/totipotent progenitor stem cells can be utilized to rapidly accumulate and maintain a perpetual pool of transgenic cell lines.

Various medium compositions were formulated (Table 1) in trials to find a robust formula that would match and ensure that the greatest majority of transgenic pluripotent/totipotent progenitor stem cells would regenerate into plants. Transformation efficiency was raised from an average of 3% to a range from about 5-30% depending on replicate (Table 4). After only one week of regeneration the media could be ranged in order of efficiency from highly efficient to lowest, as 289J#1>289J#4>289J#2>289J#3>289J>289J#5>(UCB; 289J+2 mg/l BAP) (Table 5). Medium composition 289J#4 gave the overall highest efficiency of 30.7% at the callus level. This efficiency dropped down to from about 5 to about 20% at the plantlet level.

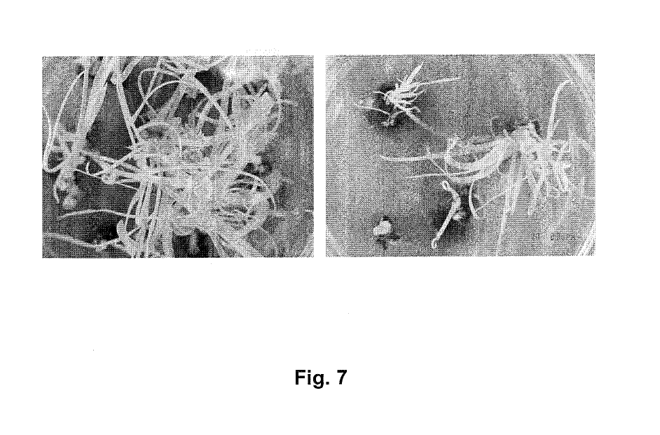

Transformation frequencies of about 12% at the plant level were obtained when sorghum was transformed via biolistics. Considering that current levels of transformation efficiency in sorghum using popular standard procedures developed by Zhao et al., (2000) obtain maximum transformation efficiencies of about 3%, techniques developed in this research represent an improvement of over 66% [(5/3×100)−100]. Counts indicated that over 99 transgenic plantlets (assumed to be clones derived from a single cell line) could be obtained per transformed immature zygotic embryo of sorghum. Some regenerated plantlets are shown in FIG. 7.

TABLE 4
Transformation efficiency at the callus level (pre-regeneration)
engendered by the introduction of a phase enriching for
embryogenesis, pluripotency, totipotency and accumulation
and maintenance of pools of transgenic cells

| | | | Percentage |
|---|---|---|---|
| | PHP 15325 | | transformation |
| | infected | No. of putative | frequency at 9-12 |
| Replicate/ | immature | events (GFP) at | weeks transgenic |
| Experiment | embryos | callus level | callus level |
| 1 | 106 | 22 | 20 |
| 2 | 83 | 12 | 14 |
| 3 | 44 | 11 | 26 |
| 4 | 114 | 28 | 24 |
| 5 | 58 | 5 | 8 |
| 6 | 50 | 2 | 5 |
| 7 | 42 | 7 | 19 |
| 8 | 52 | 15 | 28 |
| 9 | 21 | 3 | 14 |
| 10 | 13 | 3 | 23 |
| 11 | 19 | 4 | 22 |
| 12 | 971 | 252 | 26 |
| 13 | 18 | 5 | 30 |
| 14 | 48 | 9 | 18 |
| 15 | 44 | 7 | 16 |
| 16 | 959 | 239 | 25 |
| 17 | 829 | 116 | 14 |
| Standard | 20 000 | 600 | 3 |
| procedures: | (average) | | |
| No enrichment | | | |
| phase | | | |

TABLE 5
Efficiency of various media compositions for the regeneration of events
developed from the introduction of stem cell/pluripotent or totipotency
enrichment phase in the transformation procedure of sorghum

| | No. of transgenic | | Events regenerated | |
|---|---|---|---|---|
| Agrobacterium | events at callus | | to shoot stage after | Transformation |
| vector used for | level (based on | Regeneration | only one week on | efficiency at |
| transformation | GFP expression) | medium | regeneration | regeneration stage |
| PHP15325 | 107 | 289J#1 | 35 | 32.7% |
| PHP15325 | 108 | 289J#2 | 20 | 18.5% |
| PHP15325 | 103 | 289J#3 | 18 | 17.5% |
| PHP15325 | 104 | 289J#4 | 32 | 30.7% |
| PHP15325 | 109 | 289J#5 | 9 | 8.25% |
| PHP15325 | 86 | UCB | 0 | 0 |
| PHP15325 | 66 | 289J + 2 mg/L | 0 | 0 |
| | | BAP | | |
| PHP15325 | 112 | 289J | 18 | 16.1% |

The applicants have shown herein that the concept of “stem cells” in plants can be exploited to enrich for morphogenetic plasticity and competence for regeneration in sorghum and maize. The data presented further suggest that the greatest impediment to efficient sorghum transformation goes beyond Agrobacterium tumefaciens T-DNA transfer or biolistics, to encompass difficulties in efficiently proliferating few transgenic cell lines and the in vitro organization of such cells to differentiate and then regenerate transgenic plants. Introducing a highly efficient molecular and physiological step, for example enriching sorghum callus cells for pluripotent progenitor stem cells, which directly participate in meristematic shoot organogenesis, into a standard protocol of sorghum transformation should thus elevate transformation frequencies from their currently unsatisfactory low levels. A good candidate protocol for sorghum transformation into which the technique we described herein could be compatibly inserted is the widely quoted and utilized protocol of Zhao et al. (2000). Ideally, the technique and steps described herein could be inserted at stages following several rounds of selection with PPT (for example after one month of selection). In the applicants' experience, this period coincides with the stage around which losses of putative transgenic cell lines is heaviest.

The data further underscores the need to approach sorghum as a unique case meriting unique molecular approaches and attention to molecular and biochemical or physiological finer details.

To show that this approach to enrich for competence in tissue culture is versatile and likely to have positive impacts on sorghum transformation efficiency, it was also shown that in corn, this enrichment and eventual regeneration is prolific and independent of genotype (FIG. 3).

Notably, most of the published research with meristematic tissues employs already established meristems, derived from either pre-germinated seeds of young or old embryos (Bommineni et al., 1989). The present results show that deriving these meristematic tissues from undifferentiated callus is very efficient and makes it easier to viably integrate this step into current Agrobacterium and biolistics transformation protocols to alleviate difficulties associated with physical injury to cells, a slower cell cycle in sorghum, the production of deleterious phenolic compounds and the general intransigent nature of sorghum cells to maintenance and regeneration from a starting point of a few transgenic cell lines. This, combined with the ease with which callus cells are easier to handle and manipulate should ideally make positive contributions towards higher transformation efficiencies in sorghum and high throughput transformation systems.

Enrichment for pluripotent and totipotent progenitor stem cells was achieved herein by utilising the phytohormones Benzyl Amino Purine (BAP) and 2,4-Dichlorophenoxy Acetic Acid (2,4-D). In undifferentiated cells, the effect of auxins and cytokinins is thought to be synergistic: both induce the expression of cdc2 kinases and cyclins. In lateral root primordial cells, the interaction between auxins and cytokins is antagonistic: auxins stimulate and cytokinins reduce the levels of cdc2 kinase. The expression of at least one cyclin is increased by auxin. Cyclin-dependent kinases (CDKs) are specific serine/threonine kinases that control progression through the cell cycle in all eukaryotes, but their activity is regulated by association with cyclins and by specific phosphorylation/dephosphorylation events.

Many other genes and mechanisms have been shown to be operative in the formation and maintenance of meristematic tissues. These include, for example, the homeodomain protein of WUSCHEL in the regulation of cell fate; and SCARECROW in specifying, maintaining and positioning stem cells. In addition to phytohormones, molecular controls of the cell cycle must also integrate environmental signals as well. These include, among other things, molecular components, nutrients in culture medium, temperature and handling during frequent subcultures as specified in the materials and methods herein.

Some of the observations noted in this research include:
Increased effectiveness of selection because the selection phase can be extended without loss of cellular vitality and regenerability.
Aid in overcoming cell death, cell necrosis and deleterious phenolic compounds owing to a faster cell cycle.
Versatility and applicability to other crops (this technique was successfully applied to the maize genotypes GS3 and TRX—a Pioneer Hi-Bred genotype).
Engenders higher transformation frequencies, up to 30% at putative callus phase, and over 10% at plant level.
The techniques are compatible with high throughput transformation systems
Organogenesis is activated from undifferentiated callus cells and, therefore, the process is decoupled from organized tissues, thus circumventing the production of chimeric plants (as is the case with transforming organized tissues).

While the invention has been described in detail with respect to specific embodiments thereof, it will be appreciated by those skilled in the art that various alterations, modifications and other changes may be made to the invention without departing from the spirit and scope of the present invention. It is therefore intended that the claims cover or encompass all such modifications, alterations and/or changes.

The following references are included herein by reference:
Bommineni, V. R., Walden, D. B., and Greyson, R. I. 1989. Recovery of fertile plants from isolated, cultured maize shoot meristems. Plant Cell Tiss. Organ Cult. 19: 225-234.
Carvalho, C. H. S., Zehr, U. B., Gunaratna, N., Anderson, J., Kononowicz, H. H., Hodges, T. K. and Axtell, J. D. 2004. Agrobacterium-mediated transformation of sorghum: factors that affect transformation efficiency. Genet. Mol. Biol 27: 259-269.
Hansen, G. 2000. Evidence for Agrobacterium-induced apoptosis in maize cells. Mol. Plant-Microbe Interact. 13: 649-657.
McCullen, C. A. and Binns, A. N. 2006. Agrobacterium tumefaciens and plant cell interactions and activities required for interkingdom macromolecular transfer. Ann. Rev. Cell Develop. Biol. 22: 101-127.
Zhao, Z. Y., Cai, T., Tagliani, L., Miller, M., Wang, N., Pang, H., Rudert, M., Schroeder, S., Hondred, D., Seltzer, J. and Pierce, D. 2000. Agrobacterium-mediated sorghum transformation. Plant Mol. Biol. 44: 789-798.
Zhu, H., Mathukrishana, S., Krishnaveni, S., Wilde, G., Jeoung, J-M. and Liang, G. H. 1998. Biolistic transformation of sorghum using a rice chitinase gene. J. Genet. Breed. 52: 243-252.

1: A process for the production of pluripotent and/or totipotent progenitor cereal cells, the process comprising the steps of:
selecting a population of cells including undifferentiated cereal callus cells; and
culturing the undifferentiated cereal callus cells in a primary plant tissue culture medium containing at least one auxin and at least one cytokinin to produce pluripotent and/or totipotent progenitor cereal cells.

2. (canceled)

3: The process according to claim 2, wherein the progenitor cells are multiplied at a greater rate than non-progenitor cells.

4: The process according to claim 1, wherein the undifferentiated cereal callus cells are selected from the group of cereals consisting of sorghum, maize, wheat, barley, millet, rye, canola, alfalfa, triticale and rice.

5: The process according to claim 1, wherein the cytokinin is selected from the group consisting of benzyl amino purine, benzyladenine, thidiazuron, zeatin, isopentyladenine, trans-zeatin and dimethylallyladenine.

6: The process according to claim 1, wherein the auxin is selected from the group consisting of 2,4-dichlorophenoxyacetic acid, indole-3-acetic acid, picloran, naphthelenacetic acid, indole-3-propionic acid, indole-3-butyric acid, phenyl acetic acid, benzofuran-3-acetic acid and phenyl butyric acid.

7-8. (canceled)

9: The process according to claim 1, wherein the pluripotent and/or totipotent progenitor cereal cells organize into cell aggregates to form shoot apical meristematic domes and primordial shoots by a process of direct organogenesis.

10: The process according to claim 1, wherein the undifferentiated cereal callus cells are obtained from plant tissue that has been transformed with an homologous or heterologous gene.

11. (canceled)

12: The process according to claim 10, wherein the transformation step is Agrobacterium-mediated.

13: The process according to claim 10, wherein the transformation step is via biolistic bombardment.

14: The process according to claim 1, wherein the pluripotent and/or totipotent progenitor cereal cells are maintained in a state of perpetual proliferation.

15-16. (canceled)

17: The process according to claim 16, wherein the secondary plant tissue culture medium includes at least one cytokinin and/or at least one auxin.

18. (canceled)

19: The process according to claim 10, wherein the transformation frequency of the process is at least 5%, at least 10%, at least 15%, at least 19%, at least 20% or at least 30%.

20-24. (canceled)

25: The process according to claim 1, wherein the undifferentiated cereal callus cells are from scutellum tissue from an embryo.

26-29. (canceled)

30: A pluripotent and/or totipotent progenitor cell cereal cell produced by the process of claim 1.

31: A process for producing transgenic cereal cells, the process comprising the steps of:
transforming cereal tissue;
selecting from the transformed cereal tissue a population of cells including undifferentiated cereal callus cells; and
culturing the undifferentiated cereal callus cells in a primary plant tissue culture medium containing at least one auxin and at least one cytokinin to produce pluripotent and/or totipotent progenitor cereal cells.

32: Transformed progenitor cells produced by the process of claim 31.

33: A transgenic plant part, plantlet or plant obtained from pluripotent and/or totipotent progenitor cereal cells produced by the process of claim 10.
